(19) United States
(12) Patent Application Publication
(10) Pub. No.: US 2019/0022009 A1
(43) Pub. Date: Jan. 24, 2019

(54) PROCESS FOR OBTAINING A BIOPOLIMERIC HEMOSTATIC POWDER AND THE PRODUCT

(71) Applicants: FUNDACIÓN PARA LA PROMOCIÓN Y DESARROLLO TECNOLÓGICO DEL LITORAL (VINTEC), SAPEM (AR); DINT S.A., SAPEM (AR)

(72) Inventors: Julio Alberto LUNA, Pcia. de Santa Fe (AR); Federico Adrian FOOKES, Santa Fe Capital (AR); Maria Alejandra CANTEROS, Santa Fe (AR)

(21) Appl. No.: 16/039,394

(22) Filed: Jul. 19, 2018

(30) Foreign Application Priority Data
Jul. 21, 2017 (AR) AR20170102049

(51) Int. Cl. A61K 9/16; A61L 15/28; A61L 15/44; A61L 15/58; A61L 26/00; A61F 13/00

(52) U.S. Cl. CPC A61K 9/1682; A61K 9/1623; A61K 9/1652; A61L 15/28; A61L 15/44; A61L 2400/04; A61L 26/0052; A61F 13/00063; A61F 2013/00472; A61L 2300/418; A61L 15/58

(57) ABSTRACT

A method for obtaining a biopolymer hemostatic powder including adding in an reactor (1) a mixture of an organic acid (b), a combination of alcohols (b) (d) and a bioadhesive to obtain a binder solution and a step of incorporating on a base of the lower part of a fluid bed reactor (2) a polysaccharide (a) driven by an air stream injected at controlled temperature and speed; on which fluid and micro particles of the polysaccharide (a) are sprayed from the top of said fluid bed reactor (2) the binding solution and where the polysaccharide (a) is chitosan, the organic acid (b) is acetic acid, the alcohol combination (c) is alcohol and polyalcohol and the bioadhesive is polyvinyl alcohol. The product contains between 55 and 85% w/w of a polysaccharide; between 10 to 40% w/w of an organic acid; up to 17% w/w of a combination of alcohols and up to 3% w/w of a bioadhesive; whose alcohol combination is composed of 95% of an alcohol and 5% of a polyalcohol and the binder fluid is sprayed onto the polymer in an amount of between 50 and 150% p/p.

### FIELD OF THE INVENTION

The present invention includes a process for obtaining a biopolimeric hemostatic powder and the obtained product.

In order to make the present invention understandable so that it can be easily implemented, an accurate description of a preferred embodiment will be given in the following paragraphs. This description is completed with a drawing that allows to exemplify the invention without that such description and drawings can be considered, in any case, to be limiting the invention.

To better illustrate what is described here, the results of studies requested to the Universidad Nacional del Litoral are attached as well as the photographs of the clots that were obtained as a result of the tests performed.

The components referred to in the description may be selected from various equivalents without implying departing from the principles of the invention set forth in this documentation.

### BACKGROUND OF THE ART

The inventors have developed a preparation that includes a fast-acting hemostatic powder for the temporary treatment of external bleeding wounds, deep wounds, and even the bleeding of the larger arteries. The powder can be used by any person with a minimum knowledge of first aid. The preparation stabilizes hemodynamic and is effective even in the presence of anti-coagulated blood because it acts independently of the physiological coagulation process (coagulation factors).

The active principle of the hemostatic powder that is an object of the present invention is chitosan, which is a natural polysaccharide (copolymer of N-acetylglucosamines and glucosamine), biodegradable, biocompatible, bioadhesive, and non-toxic. It is obtained by the partial alkaline des-N-acetylation of chitin, under the heading are included a series of polymers of different molecular weights (50 to 2000 KDa) and deacetylation degree (70-98%), whose versatility of properties is constituted in materials of different features.

The chitin is a polysaccharide that is part of the exoskeleton of crustaceans and insects and is also found in some mushrooms. After the cellulose, it is the second most abundant polymer on earth, which gives it a relevant interest as a source of biocompatible materials. Among the many uses of chitosan can be highlighted the treatment of wastewater, the use of in food technology, agriculture, and others.

Its importance most common in pharmacology refers to its action as a binder and disintegrator, but also acts as a coating polymer and as a matrix for controlled release. It has an important antimicrobial action, it is a facilitator of the trans-epithelial transport and an effective healer.

Some of the properties of chitosan have been known since 1983, among them, accelerating coagulation in vitro, independently of the natural coagulation process, an effect that has been attributed to the interaction with the cell membrane of erythrocytes.

In its granular form, this agent interacts directly with red blood cells and platelets forming a pseudo-clot that acts as a mechanical barrier and generation nucleus.

The same can be degraded in vitro by lizosima, papain, and pepsins, among others. Its biodegradation leads to the release of variable and non-toxic oligosaccharides chain, which subsequently can be incorporated into the metabolism of glycosaminoglycans, glycoproteins, or excreted.

In the prior art, there are numerous products that incorporate the aforementioned active principle and with similar benefits. Thus, they were approved for use and distribution in the United States territory and the European Union, among others, HemCom Patch® PRO, Syvek® Patch, HemCom® Bandages PRO, ChitoFlex® PRO Haemostatic Dressings, ChitaGauze® PRO, Chitodine ® (HemCon Medical Technologies, Inc); ChitaSeal® (Abbott vascular devices); Traumastat® (Ore-Medix); ExcelArrest® (Hemostasis LLC Co); Celox™ (MedTrade Products Ltd), these products are presented in the form of patches, bands, gauzes, dressings, foams, and granules.

In summary, it can be summarized that the properties of the claimed product are mainly as haemostatic and antimicrobial.

In the natural process for loss of blood, there is a reflex reaction of the organism that produces a spasm by which it contracts the blood vessels and produces an agglomeration of platelets in the affected area so that a plug is produced while accelerating the growth of the fibrous tissue.

In small vessels, the work is carried out by the platelets in conjunction with the Golgi apparatus. The platelets are actin molecules, myosin, and thromboestein and are combined with residues of endoplasmic reticulum, mitochondria-ATP, PGs, the fibrin stabilizing factor, and the growth factor. The membrane adheres to the damaged endothelium and produces an increase in the volume of the contractile proteins to then contract and become sticky from the collagen that they secrete, causing a chain reaction where the enzymes activate more platelets.

The substances that promote coagulation are known as procoagulants and lead to a series of complex chemical reactions that, in the presence of ionic calcium, activate the prothrombin that becomes thrombin and this polymerizes fibrinogen molecules to fibrin. The prothrombin binds to the platelet receptors attached to the damaged tissue.

As it emerges from the prior art, artificially the coagulation time is shortened by the addition of various substances such as those that make up the CELOX brand products and the hemostatic powder of the present invention.

The products of the prior art, as well as the one proposed here, are based on the difference in electrical charge that determines the union between red cells of negative charge with the granules of the product, of positive charge.

The inventor is aware of WO 2012123728 A3 which relates to a hemostatic material including a haemostatic material and a bioadhesive agent. The hemostatic agent has an effective control over the bleeding with a reduction of the compression period with the TCCC orientation of one minute compared to the three minutes of compression using a hemostatic dressing.

The haemostatic agent of the cited document is selected from regenerated oxidized cellulose; kaolin; gelatine; calcium ions; zeolite; collagen; chitosan, and its derivatives.

It is further disclosed that the stated hemostatic agent may include a chitosan salt or a mixture of chitosan salts selected from chitosan acetate; chitosan lactate; chitosan succinate; chitosan malate; chitosan sulfate, and chitosan acrylate.

The hemostatic agent may be a granulate, a powder, short fibers, a liquid, a gel, or a spongy tissue and the pH of the stated hemostatic agent is between 3.5 and 8.

For its part, the bioadhesive agent is selected from at least one of the following: carbomers; polyvinyl alcohol; polyvinyl pyrrolidone; 2-acrylamido-2-methylpropane sulfonic acid; an acrylic acid polymer with a molecular weight of at least 50000 G/mol cross linked with di-vinyl glycol or polyacrylic acid salts cross-linked with di-vinyl glycol.

The document US 0186851 A1 of the year 2009 is analyzed from which the first claim refers to a hemostatic powder composed of a chitosan salt together with at least one material from the group comprising chitosan acetate, chitosan lactate, succinate of chitosan, chitosan malate, chitosan sulfate, and chitosan acrylate.

In the mentioned document, the procedure to obtain the product includes dissolving the chitosan acetate in a hydro alcoholic acid medium to then evaporate the solvent maintaining a temperature of 60° C. to finally process the dry particles in a mill to obtain the desired granule size.

In the present document, a procedure is disclosed that allows to significantly reduce the coagulation time necessary to control the bleeding, improving even the coagulation times of previous art products.

According to the tests carried out by the inventor, the reduction of the times is greater than 50% in relation to the times required by the known coagulants.

The preparation obtained with the procedure that is revealed is a hemostatic preparation of quick action that can be used by people who have minimal knowledge of first aid.

The product obtained allows to stabilize hemodynamic, it is not a pro-coagulant, and is effective even in blood treated with anticoagulants because it acts independently of the physiological coagulation process (coagulation factors). The product does not generate heat, so it does not cause burns in patients or health personnel; although it alters the pH in the place of application, it is not enough to be corrosive in the contact zone, and it is biocompatible. Once it reacts, forms a soft nucleus, slightly sticky that is easily manually removable, while the remaining material is washed with saline solution. In case it could absorb traces, it is eliminated by the normal metabolic because it is biodegradable.

The invention that is disclosed includes a step where the components are placed in a fluid bed reactor and between the components, unlike the pre-existing, an inert material is not included.

### SUMMARY OF THE INVENTION

Basically, the present invention includes a process for obtaining a biopolymer hemostatic powder having a plurality of steps including placing the components in a reactor and stirring them to obtain a binder solution and entering by spray and from the top end of a fluid bed reactor the solution obtained by injecting from the bottom of the same air with micro particles of a polysaccharide.

The process includes sifting the mixture extracted from the fluidized bed reactor by sending the particles that passed the mesh to a packaging equipment and those that did pass to a mill and from there again to the fluidized bed reactor repeating the cycle.

Finally, the fractionated, packed, and sealed product is sterilized by radiation.

The product obtained with the described method includes a polysaccharide, an acid, alcohols, and a bioadhesive.

### BRIEF DESCRIPTION OF THE DRAWINGS

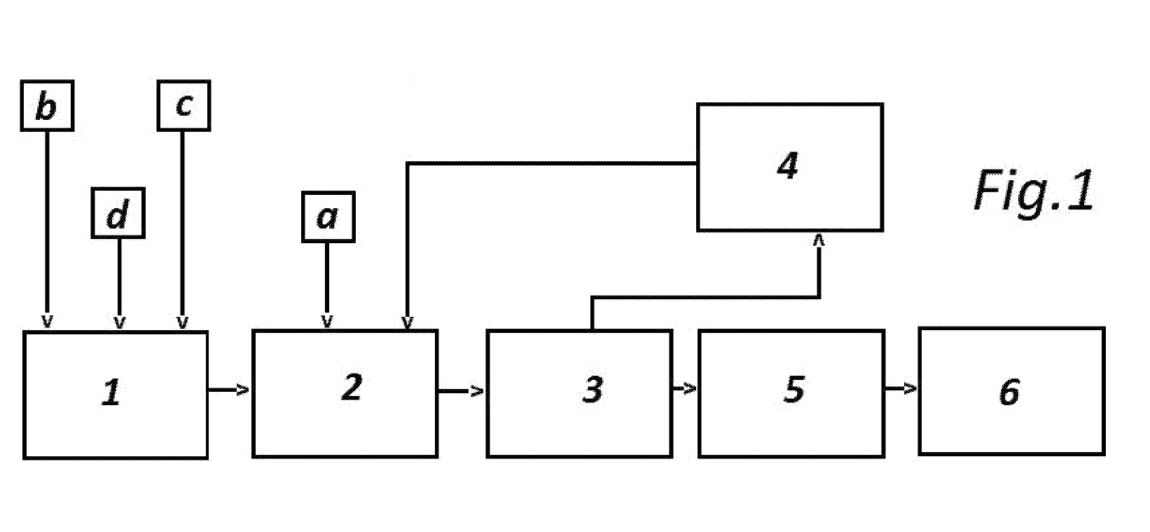

In order to achieve a better understanding of the object of the present documentation is shown in FIG. 1, and the only one, a flow diagram in which the various components that make up the invention disclosed are represented.

### DETAILED DESCRIPTION OF THE INVENTION

In the accompanying figure, references have been incorporated that allow individualizing the various parts and components of the invention.

In the figure, the same references indicate equal parts or components.

It is individualized accordingly, with the number -1- a mixing reactor; with the number -2- a fluid bed reactor; with the number -3- a sieve; with the number -4- a mill; with the number -5- a packing equipment and with the number -6- a sterilizer.

With the letter -a- a polysaccharide or polymer is indicated; with the letter -b- an acid is indicated; with the letter -c- a combination of alcohols is indicated and with the letter -d- a bioadhesive.

### Functioning

Once established, the different components of the developed invention to explain their nature, they are then complemented with the functional and operational relationship of the same and the result they provide.

The process for obtaining a biopolymeric hemostatic powder that is disclosed is integrated with a series of steps comprising a step of incorporating acids -b-, alcohols -c-, and bioadhesives -d- in a mixing reactor -1- with stirring, where mix is to form a binding solution or “binder”.

In another step, the main process of haemostatic powder elaboration is applied, which is carried out using fluid bed technology.

In this step, the fluidification of a powder is carried out using two fluids. One of these fluids is the binder solution obtained which is sprayed from the top part of a fluid bed reactor -2- and the second fluid is an air current ascending that is injected from the lower part of the stated fluidized bed reactor (2) passing through a perforated base dragging micro particles of a polysaccharide powder (a) deposited on it.

The air injected at a controlled flow and temperature and containing the micro particles of the polysaccharide (a) meets the micro droplets of the binder solution sprayed from the top.

This binder solution is composed of an acid (b), a combination of alcohols (c) and a bioadhesive (d), and fluidizes the micro particles of the polysaccharide (a) powder that is granulated.

In this step, the polysaccharide (a) must be biocompatible and biodegradable, selecting the chitosan; the acid (b) is acetic acid; the alcohols (c) result in a combination of an alcohol and a polyalcohol and the bioadhesive (d) is a polyvinyl alcohol.

The method also comprises a step of including in the stated process a sieve -3- in which the mixture discharged from the fluid bed reactor (2) separates the screened particles from those having the appropriate granulometry to overcome the mesh of the sieve (3) and those that do not have it.

Thus, in another step, those particles that have not passed the mesh of the sieve (3) are derived to a mill -4- from which, with a smaller granulometry, are reinjected into the fluid bed reactor (2).

On the other hand and in a new step, all those particles that have overcome said mesh are derived towards a packaging equipment -5-.

The particles re-injected in the fluid bed reactor (2) together with new particles that are obtained from the mixing reactor (1) are derived to the sieve (3) where a new selection is made. This circuit is repeated until the entire mixture passes through the mesh of the sieve (3) to the packaging equipment (5) where it proceeds to the dosing, packaging, and sealing of envelopes containing the product.

Alternatively, the packaging can be carried out in powder applicators that are closed with a lid and pack.

When the envelopes or applicators containing the product leave the packaging equipment (5) they are derived to a sterilizer -6- where they are sterilized with gamma rays, ready to be used.

As it was said, in the preferred embodiment, the polysaccharide (a) of the aforementioned characteristics is chitosan, whose cationic character increases when combined with the acid (b). This increase in the cationic character increases the hemostatic power by improving the interaction between the polysaccharide (a) and the red blood cells, being that this is one of the necessary steps for the induction of coagulation.

The use of acid (b) acidifies the mixture so that an analysis of the clot formed will give a pH value of 5.23±0.1 value that is acceptable for the use of this type of product.

As it emerges from the analysis that are accompanied, in-vitro coagulation studies have been carried out with the product obtained and with a similar product that exists in the market and which is marketed under the trademark Celox.

The methodology used was as follows:

The coagulation time of rabbit blood freshly extracted from the ear veins was analyzed or by cardiac puncture according to the procedure published by Johnson L, Ranfield J. & Hardy C (Johnson L 2008).

To 7 mL of the extracted rabbit blood was added 1 g of the samples to be evaluated, which in the case were the product of the CELOX registered trademark and the hemostatic powder obtained with the procedure disclosed in this documentation and a second sample of 7 mL of rabbit blood anti-clotted with heparin (90.9 units USP/mL).

Both samples were subjected to rotation and agitation with oscillation in a 180° arc with a frequency of approximately of 1.5 sec. to ensure an optimal mix by recording the time that it took for the clots and pseudo-clots to form and then photograph them.

At all events, in both cases, four control samples were taken.

In all the experiences, the hemostatic aggregates significantly reduced the coagulation times (p<0.001) and all the clots formed presented a very good adhesion to the container that contained them, a desirable characteristic since by achieving the permanence of the clot in the place of trauma, facilitates the transfer of the patient when it must be mobilized.

The hemostatic powder obtained with the method of the present document presented better coagulation times using blood without anti-coagulants with respect to the CELOX-brand haemostatic powder used as a control.

But no significant differences were found in the coagulation times when anti-coagulated blood was used compared to that without anti-coagulate (p<0.05). This represents a remarkable property, since it demonstrates the independence of the normal coagulation mechanism (Cascade of factors), greatly expanding the spectrum of clinical use to be applied to patients with coagulation problems.

As stated above and taking into account that the procedure uses acid (b), it was necessary to determine the pH of the clots formed in order to determine the compatibility with its use in humans and animals.

To carry out this trial, the following methodology was followed:

The pH of the formation of the pseudo-clot “in vitro” was determined for which proceeds as revealed in previous paragraphs to the addition of 1 g of the hemostatic obtained according to what is revealed in the present application to 7 ml of anticoagulated blood, rotation was applied, agitation, and frequency to guarantee an optimal mixture and the pH is recorded with a Hanna HI-9124 pH meter after 20 minutes of the hemostatic addition.

The results of the in vitro hemostasis tests carried out show that the effectiveness of the hemostatic powder obtained with the procedure of this documentation is superior to the commercial product CELOX brand elaborated by Medtrade Products Ltd. used for the comparison. Thus it has been determined that the blood clotting time decreases between 40 and 64 seconds, that is, it is between 56 and 68% faster.

The invention also comprises a product including a biopolymer hemostatic powder obtained by processing a polysaccharide (a) to obtain a salt by including it in an aqueous solution of an organic acid (b) applied in micro drops; both components being entered in a fluid bed reactor (2).

As it was said, in the fluidized bed reactor (2) a current of air is injected that drags the polysaccharide (a) deposited in a sieved base. This air current maintains the polysaccharide (a) in suspension and over it is injected a cloud of micro droplets of a solution that includes an organic acid (b), a combination of alcohols (c), and a bioadhesive (d).

The polysaccharide (a) is chitosan and in the solution the organic acid (b) is acetic acid; the alcohol combination (c) is composed of an alcohol and a polyalcohol and the bioadhesive (d) is polyvinyl alcohol.

The solution that is sprayed is integrated with between 10 to 40% acetic acid; up to 15% alcohol; up to 2% polyalcohol, and up to 3% polyvinyl alcohol.

This solution is sprayed on the polymer in an amount of between 50 and 150% w/w.

In this way, one of the possible sequences of steps leading to the invention and the manner in which it operates is described, and the documentation is supplemented with the synthesis of the invention contained in the clauses claiming to be added below.

Follow the claims on page 10

Having described and determined the nature of the invention, its scope and the manner in which it can be put into practice in its fundamental idea, the following is declared as an invention and of exclusive ownership:

1. A method for obtaining a biopolymer hemostatic powder, the biopolymer hemostatic powder includes a polysaccharide, an acid, at least one alcohol, and a bioadhesive, the method comprising the steps of:
stirring in an admixture reactor, an organic acid, a mixture of alcohols, and a bioadhesive to obtain a binder solution;
injecting from a base of a lower part of a fluid bed reactor, an air stream having a polysaccharide at controlled temperature and speed; and
spraying from a top part of the fluid bed reactor, the binding solution to form particles;
wherein the polysaccharide is chitosan, the organic acid is acetic acid, the alcohol combination includes an alcohol and a polyalcohol, and the bioadhesive is polyvinyl alcohol.

2. The method according to claim 1, wherein the binder solution is sprayed on the polysaccharide in an amount of between 50 and 150% w/w.

3. The method according to claim 1, wherein the organic acid lowers the pH in the clot to a value of 5.23±0.1

4. The method according to claim 1, wherein the stirring step is a bobbing delimited in an arc of 180 ° with an approximate frequency of 1.5 seconds.

5. The method according to claim 1, further including the steps of:
sieving the sieve the particles by using a mesh of between 75 and 1000 pm:
packing the particles crossing the mesh, milling the particles not crossing the mesh and then returning to the fluidized bed reactor repeating the cycle until all the particles pass through the mesh.

6. The method according to claim 54, further including the stop of radiating the packaged particles with a gamma radiation of the order of 25 to 35 kGy -is--apOied in a the sterilizer.

7. A biopolymer hemostatic powder, obtained with the process of claim 1, wherein the biopolymer hernostativc power -contains between 55 and 85% w/w of a polysaccharide; between 10 to 40% w-/-w of an organic acid; up to 17% w/w of a combination of alcohols and up to 3% w/w of a bioadhesive;
whose alcohol combination is composed of 95% of an alcohol and 5% of a polyalcohol and the binder fluid is sprayed onto the polymer in an amount of between 50 and 150% p/p.

8. The powder according to claim 7, wherein in the binder solution the organic acid is acetic acid, the combination of alcohols with an alcohol and a polyalcohol, and the bioadhesive is polyvinyl alcohol, and the polysaccharide is chitosan. US Application No. New Docket No. 1149.001

9. The powder according to claim 7, wherein the combination of alcohols includes up to 15% of an alcohol and up to 2% of a polyalcohol.
